(19) United States
(12) Patent Application Publication
(10) Pub. No.: US 2010/0010379 A1
(43) Pub. Date: Jan. 14, 2010

(54) DEVICES AND METHOD FOR MONITORING THE FORM OF THREE-DIMENSIONAL OBJECTS

(76) Inventors: Danilo Emilio De Rossi, Pisa (IT); Federico Lorussi, Pisa (IT); Mario Tesconi, Pietrasanta (IT); Alessandro Tognetti, Lucca (IT)

Correspondence Address: BROWDY AND NEIMARK, P.L.L.C.;624 NINTH STREET, NW, SUITE 300, WASHINGTON, DC, 20001-5303, US

(21) Appl. No.: 11/721,749

(22) PCT Filed: Dec. 13, 2005
(86) PCT No.: PCT/EP2005/056749

§ 371 Date: Aug. 7, 2009

(30) Foreign Application Priority Data
Dec. 14, 2004 (IT) FI2004A000264

(51) Int. Cl. A61B 5/103

(52) U.S. Cl. 600/587

(57) ABSTRACT

Devices for monitoring the deformation of three-dimensional objects thanks to the presence of sensors integrated in the device are described.

### FIELD OF THE INVENTION

The present invention relates to the field of monitoring three-dimensional bodies.

### PRIOR ART

The problem of determining the form of deformable or spatially articulated three-dimensional objects is difficult to solve using the methods available in the prior art. The solution to this problem also comprises the movement (in terms of the evolution of the form in time) of three-dimensional objects (for example in the field of reconstruction of human posture and body movements on the basis of body kinematic variables). Some attempts to solve the problem described above exist in the prior art. For instance, some instruments use electromagnetic sensors, strain gauges, or cameras and markers.

Generally speaking, the existing devices are too expensive and too bulky (for example due to the presence of mechanical constraints and metal wires) and are unsuitable for monitoring non-conventional objects such as highly deformable elastic objects. This is because since the mechanical parts that are used are often non-stretchable or even rigid, they may get in the way of certain movements or cause mechanic artefact. Furthermore the existing devices are not universal in that they work differently when used on objects even with slightly different morphology. One example regards the field of movement analysis using wearable systems to measure joint angles, in which angular sensors (generally referred to as electrogoniometers) are applied to ordinary garments in correspondence with the main joints (list) of the human body in order to measure the angular variables. In this case even slight morphological differences (different joint lengths) can mean that the sensors are positioned incorrectly and must therefore be repositioned each time a different object is studied. Another problem regarding data acquisition is the phenomenon of cross-talk between the sensors. Considering elastic, non-rigid or even just flexible substrates, the transmission of forces and strains along the actual surface alters the mechanical properties of the substrate even in areas distant from the point in which the strain is applied, altering the value returned by sensors distant from the point in which the force is applied. In particular, with reference to biomechanical analysis, joint movements detected by sensors on a deformable substrate may alter the value of sensors that are spatially close to other immobile joints. In general when monitoring the form of three-dimensional objects, dependence between a specific sensor and degree of freedom is never achieved.

From that stated above the advantages of systems with integrated sensors to monitor mechanical deformation fields applied to surfaces are clear.

In particular, when determining body kinematic variables, comfort is an essential requirement for non-invasive use over long periods of time, while more generally adherence to a specific form is essential for determining the actual form.

### BRIEF DESCRIPTION OF THE DRAWINGS

FIG. 1 illustrates a three-dimensional model for defining the position and number of the sensors on the substrate;

FIG. 2 illustrates a template obtained starting from the model in FIG. 1 in relation to a hand (glove);

FIG. 3 (A-D) illustrates the various steps in the process of applying the elastomer to the substrate;

FIG. 4 shows the arrangement of the sensors on a knee-band;

FIG. 5 illustrates the lumped electrical model of the circuit that is printed on the substrate;

FIG. 6 shows the electrical circuit illustrating the method of data acquisition by the sensors.

### DESCRIPTION OF THE INVENTION

The present invention solves the problems described above with devices with integrated sensors that are (smaller) more handy, wearable comfortable than traditional systems with applied sensors.

The invention also relates to a production process for producing the aforesaid devices that allows complex topologies to be constructed on flexible media to ensure the possibility of fine sensing of specific areas of the surface being studied, even using redundant solutions, that is using more sensors than the number of variables to be determined. The independence of the morphology of the surface studied if the system is used to monitor form and movement (meaning the variation in the form in time) of different objects is assured by the redundancy of the sensorial systems. The possibility of implementing elastic and flexible interconnections means there is no mechanic noise on the movement or on the mutual positions of points of the substrate eliminating restraints on distances. Finally the phenomenon of cross-talk between sensors on the elastic substrate is resolved and used to ensure that the sensitive system operates independently of the specific type of use.

The present invention also relates to a method for monitoring the deformation of three-dimensional bodies.

The devices according to the present invention comprise a flexible substrate (stretchable and non-stretchable, preferably fabric) provided with sensors which are applied by spreading it with networks of sensors and elastic interconnections consisting of electrically conductive elastomers that have piezoresistive effects if mechanically stimulated.

The present invention thus offers the following advantages:
the sensors are integrated in the substrate, so that the form assumed by the actual substrate and/or the strains that are applied can be known and the system is smaller than traditionally applied sensing systems. If the systems are used to determine body kinematic variables, the wearability and non-invasiveness of the garments is assured, while when used to analyze forms, the adhesion to the unknown profile to be determined is assured.
the use of integrated elastic connections reduces the noise or mechanical constraints due to traditional non-stretchable connections.
the possibility of creating redundant sensor systems means the devices can be used to detect positions and forms even on morphologically diverse objects/subjects. If used to monitor body kinematic variables, posture can even be monitored on physically diverse subjects.
a number of variables can be determined with the desired precision thanks to the use of the desired geometries.

According to the invention all the sensors take part in monitoring the form and/or movement. In other systems, this phenomenon, called “cross-talk”, is considered a form of disturbance to be eliminated, whereas with this method it is used to improve the sensitivity accuracy of the entire system.

According to the invention, the sensors are not localized in the substrate, but are spread evenly, so that thanks to the above-mentioned redundancy there is always a set of sensors capable of monitoring the movement and form being studied.

On the surface of the substrate the devices according to the present invention have a conductive elastomer that adheres to the actual substrate according to predefined patterns obtained using specific templates.

The substrates according to the present invention may be fabrics consisting of natural or artificial fibers; they are preferably elastic fabrics, since the elastomer sensors applied thereto can function when stretched (strain gauge). Specific sensor configurations on both sides of a flexible substrate in which the values returned by sensors corresponding geometrically and specularly in relation to the actual substrate are read differentially, also allow forms to be determined even for non-stretchable substrates only considering the deformation (elongation of one side and compression of the other) of the applied elastomer.

The conductive elastomer may be a commercial product or an experimental product suitable for the specific purpose. In particular, intrinsic conductive polymers (such as polypyrrole, polyaniline and their derivates ) or loaded (with carbon, graphite or metal powders) polymers (such as silicon, natural rubber or polyurethane) are preferable.

Elastosil LR 3162 A, B© has been found to be particularly suitable. This material comprises two components that are mixed at the time of use with the addition of an appropriate solvent and has good mechanical and electrical properties and fast vulcanization after which it acquires a rubbery consistence.

The templates are made by means of a vector drawing, using any graphic program, starting from a drawing of the arrangement of the sensors on a virtual model so that all the factors necessary for their construction can be taken into consideration, for instance: the space available on the system, the maximum current that can be sent to the system in view of the applicable laws and also considering the specific application for which the sensors are to be used. From the analysis of these factors the negative of the drawing of the desired template is obtained on a scale of 1:1; the vector drawing obtained in this way can be used by an electronically controlled machine, using a laser cutter, to copy the drawing by cutting it onto a sheet preferably of adhesive material, since the template must be glued to the substrate.

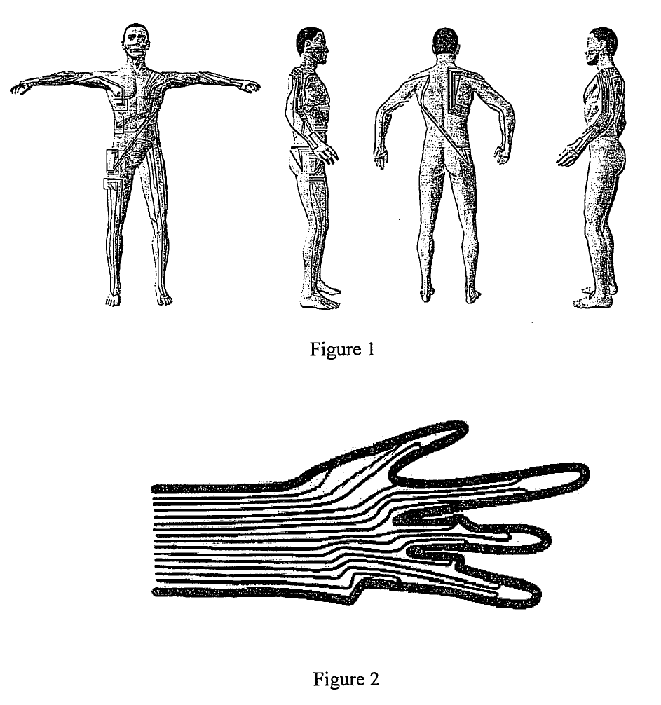

The initial drawing is preferably done on three-dimensional virtual models using commercial software packages, as shown for example in FIG. 1 in which the desired model is a human body, in which the sensors are marked.

FIG. 2 shows an example of the template obtained from the virtual model, limited to a hand of the subject, that will be applied to a glove; note that the black lines in the drawing represent the lines cut by the laser on the sheet of adhesive material as described above.

Once the sheet on which the desired template has been cut has been placed in position on the substrate, the elastomer mixture is applied to the substrate by spreading it evenly over the template so that it can be deposited along the previously made cuts.

The substrate and the template coated in the mixture are then placed in an oven at an appropriate temperature and for an appropriate time to vulcanize the elastomer and enable it to adhere to the substrate. This operation may, for example, be performed at a temperature of approximately 120° C. for approximately 15 minutes. The sensing substrate is then removed from the oven and left a few minutes to allow the mixture and template to cool, after which the template is removed from the substrate; in this way the substrate is only conductive in the parts left clear by the template.

For example, if the substrate is a fabric; the sensing fabrics thus obtained have the following important properties: non-invasive, comfortable and ensuring perfect adherence, reducing slippage between the monitoring system and the object on which it is worn to a minimum.

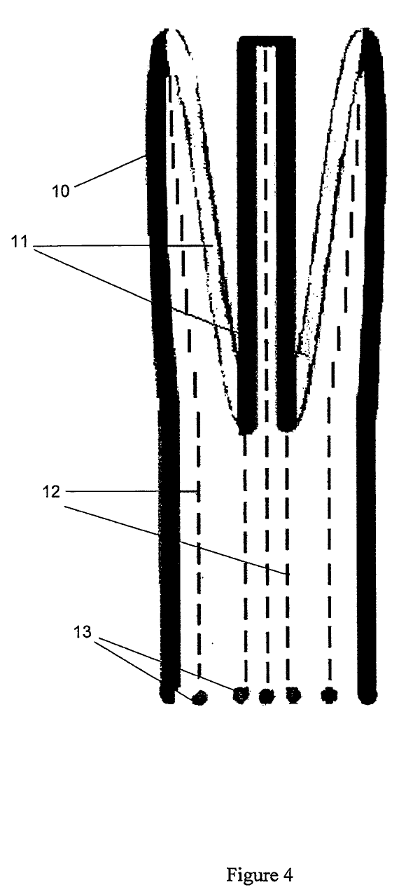

Furthermore the same material can be used to make the sensors and the electrical connections. This is an advantage in terms of the wearability of the device because there is no need for any electrical wires, to connect the sensors to the electronic acquisition system, which could obstruct certain movements. In this way, the connections to the acquisition electronics are only made to the periphery of the device in the spots (13) in FIG. 4. As far as the sensing knee-pad is concerned, for example, there are no metal wires across the joint, which could obstruct movements or create noise and motion artefacts

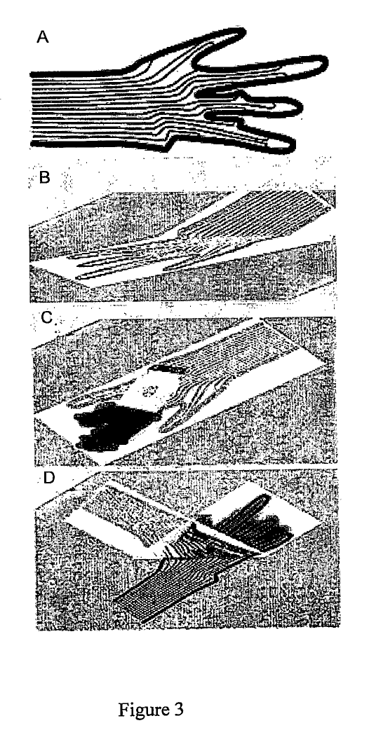

FIG. 3 illustrates the various stages of the elastomer application process, as described above.

FIG. 4 illustrates, by way of example, the application of the elastomer on a knee-band capable of detecting the position and movements of the knee joint; in this drawing the profile of the applied elastomer is visible, with the relative template obtained from the analysis of the virtual model. In this specific example the sensors are all connected in series if considering the segments responsible for picking up the signal open (this hypothesis is verified by the specific characteristics of the acquisition electronics).

The profile of the applied elastomer in this case is shown by the line 10, the sensors are shown by the lighter sections 11 of the line 10, and the wires for the connections to the acquisition electronics are shown by the broken lines 12. Note that the sensors 11 actually consist of a section of elastomer 10 between the wires for connecting the actual sensors to the acquisition electronics, the points of interconnection between the wires 12 and the acquisition electronics are indicated by the spots 13. The wires 12 and the spots 13 are also made of the same loaded elastomer.

In this particular example all the sensors are connected in series provided that no current passes along the broken lines. By supplying a constant current to the series, the values obtained by each of the various sensors can be read separately. In order that practically no current flows along the broken lines used to read the signals, instrumentation amplifiers with high input impedance are used in the first acquisition stage (FIG. 6). In this way the voltage drops on the branches R are negligible. The voltages measured by the measuring instruments (in FIG. 6 these are voltmeters due to their very high input impedance) are thus equal to the voltages on the sensors S regardless of any variation in the electrical resistance of the connections during elongation of the fabric.

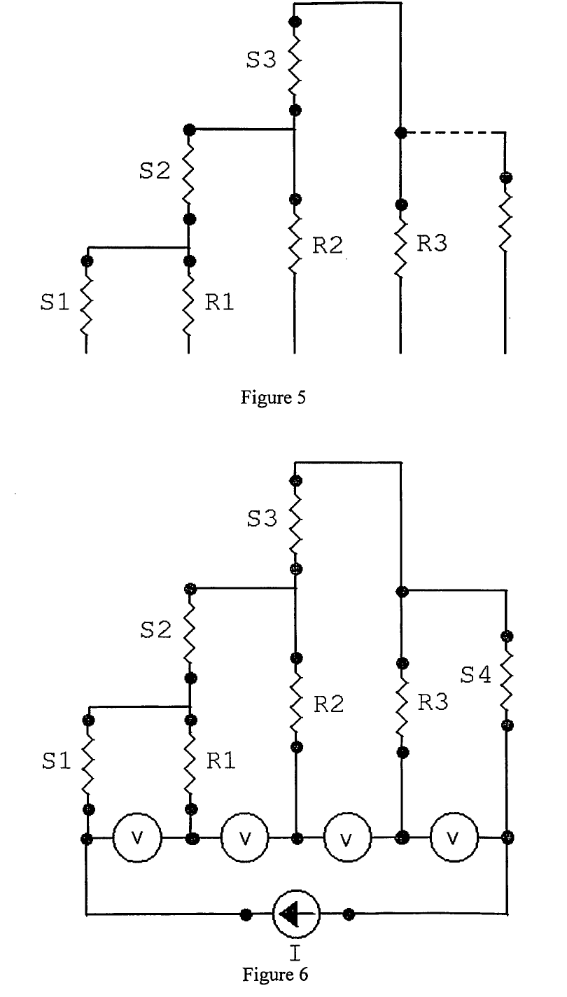

To facilitate the description of this invention, FIG. 5 shows the schematic electrical model of how the sensors applied to the fabric work according to the invention, where S₍₁₋₃₎ are the sensors in series and R₍₁₋₃₎ are the wires that connect the sensors to the acquisition electronics.

The devices according to the invention, in which the flexible substrate consists of a fabric, may be used to make garments to monitor movements or detect other kinematic or postural variables.

In addition to the fabrics described above the invention also clearly relates to the garments manufactured using said fabrics, or garments in which the sensing system according to the invention is applied to ready-made garments.

Considering the specific use for studying movements of the knee joint, a knee-band, to which the elastomer has been applied in the form illustrated in FIG. 4, is worn by the user on the knee being studied. A microampere current is supplied to the elastomer 10 (in a way that is not illustrated in the drawing) by a constant current generator (not illustrated in the drawing). When the knee is bent, in the section of elastomer 10 that constitutes the sensor 11 there is a difference in potential that is measured at the interconnections 13, and from said difference in potential the angle of flexion-extension and, where applicable, the angle of rotation of the knee can easily be measured.

When a sensing system like the one described here is used the mechanical configuration of the substrate can be linked to the state of the sensors that are present. In this way a given set of configurations (identified by the sensor states) or movements (identified with the evolution of the sensor states in time) can be recorded (stored in electronic form). These configurations or movements can then be recognized by the sensing system each time they occur.

It is also possible to refine the data acquired by the sensors by adjusting the actual data to reduce the transients in the signals the duration of which is closely linked to the type of material that is used.

Finally, a map can be drawn up of a set of configurations in a general position in the set of possible sensor values and then a map interpolation be carried out to reconstruct the exact value of the variables that characterize the mechanical configuration in correspondence with configurations of values returned by the sensors. The importance of mapping and interpolation is the solution to the problem of cross-talk, the independence from the position of the sensors or the independence from the morphology in reconstructing the configuration in terms of mechanical or movement variables. The overall map of all the variables (all the mechanical variables that characterize the configuration of the substrate and all the sensors simultaneously) considers and interprets variations in values returned by sensors geometrically distant from the points at which the strain is applied that modify the form of fields of deformation.

1-14. (canceled)

15. Device for monitoring the form of a three-dimensional object or mechanical deformation field, comprising
a flexible substrate;
a plurality of sensors being provided on the substrate; and
wires provided to connect the sensors with acquisition electronics;
wherein the sensors are made of electrically conductive elastomers having piezoresistive effects if mechanically stimulated, wherein the plurality of sensors comprise groups of sensors being formed as consecutive portions of a conductive strip of said conductive elastomer material, wherein the consecutive portions are defined through the wires provided on the substrate contacting the strip, and wherein the wires are made of electrically conductive elastomers.

16. Device according to claim 1, wherein the flexible substrate is extendible.

17. Device according to claim 1, wherein the sensors are distributed on the substrate in a uniform manner and according to the desired topology.

18. Device according to claim 1, wherein the wires are distributed on the substrate according to the desired geometries using appropriate templates.

19. Device according to claim 1, wherein the flexible substrate consists of a fabric in natural or artificial fibers, preferably elastic fibers.

20. Device according to claim 1, in which said elastomers are chosen from the group of elastomers comprising: intrinsic conductive polymers (such as polypyrrole, polyaniline and their derivates) or loaded (with carbon, graphite or metal powders) polymers (such as silicon, natural rubber or polyurethane).

21. Device according to claim 6, in which said elastomer is Elastosil LR 3162 AB, manufactured by Wacker Ltd.

22. Template for the production of a device for monitoring the form of a three-dimensional object or mechanical deformation field, the device having a flexible substrate, a plurality of sensors being provided on the substrate and wires provided to connect the sensors with acquisition electronics; the template comprising a sheet of adhesive material on which a vector drawing is printed, starting from a drawing of the arrangement of the group comprising sensors and wires on a virtual model so that all the factors necessary for their construction can be taken into consideration.

23. Process for preparing a device for monitoring the form of a three-dimensional object or mechanical deformation field using a template, comprising the steps of:
cutting the desired template onto an adhesive sheet that is placed on the substrate in which the sensors are to be integrated;
application of the elastomer solution to the fabric to be treated by spreading it evenly over the template so that it can be deposited on the substrate along the cuts in the template;
placing the coated substrate and template in an oven at a temperature of approximately 120° Celsius for approximately 15 minutes to achieve the evaporation of the solvent, the vulcanization and subsequent adhesion of the elastomer to the substrate;
cooling the mixture and template; and
removing the template from the substrate.

24. Garment for monitoring postural data, provided with a device with sensors, the garment especially being a cloth, glove, stocking, sock, etc.

25. Method of measurement using a device for monitoring the form of a three-dimensional object or mechanical deformation field, the device comprising a flexible substrate, a plurality of sensors being provided on the substrate, and wires provided to connect the sensors with acquisition electronics, wherein the sensors are receiving a constant current from a constant current generator, whereas the wires are connected to high impedance amplifiers, so that every sensor can be read separately.
